(19) United States
(12) Patent Application Publication
(10) Pub. No.: US 2025/0322936 A1
(43) Pub. Date: Oct. 16, 2025

(54) ARTIFICIAL INTELLIGENCE TECHNIQUES FOR GENERATING A PREDICTED FUTURE IMAGE OF A WOUND

(71) Applicant: Solventum Intellectual Properties Company, Maplewood, MN (US)

(72) Inventors: Muhammad Jamal AFRIDI, Woodbury, MN (US); Subhalakshmi M. FALKNOR, Woodbury, MN (US); Vahid MIRJALILI, Ramsey, MN (US); Kuangxiao GU, North Bend, WA (US); Brian D. LAWRENCE, Chandler, TX (US)

(73) Assignee: Solventum Intellectual Properties Company, Maplewood, MN (US)

(21) Appl. No.: 18/870,157

(22) PCT Filed: Jun. 5, 2023
(86) PCT No.: PCT/IB2023/055761

§ 371 Date: Nov. 27, 2024

Related U.S. Application Data
(60) Provisional application No. 63/351,954, filed on Jun. 14, 2022.

(51) Int. Cl. G16H 30/40; G06T 7/00; G16H 50/20

(52) U.S. Cl. CPC G16H 30/40; G06T 7/0016; G16H 50/20; G06T 2207/10016; G06T 2207/20081; G06T 2207/30004

(57) ABSTRACT

An example system includes processors configured to: obtain image capture data for a sequence of one or more images representative of an appearance of a wound at a corresponding image capture time, each of the images separated by a sampling time interval between the image and a next image, pass the image capture data for the sequence of images through a machine learning model trained to generate image data representing one or more predicted images of the future appearance of the wound at a corresponding future time wherein a prediction time interval between the future time and a capture time of a last image of the sequence of images is greater than each of the sampling time intervals, and output the image data representing the one or more predicted images of the future appearance of the wound.

### CROSS-REFERENCE TO RELATED APPLICATIONS

This application claims the benefit of priority to U.S. Provisional Application No. 63/351,954, filed on Jun. 14, 2022, which is incorporated herein by reference in its entirety.

### BACKGROUND

Many real-world processes, especially those of chemical and biological nature, evolve slowly over time. For example, a wound may take several weeks to fully heal, depending on the nature of the wound, the size of the wound, and the treatment used during the healing period.

### SUMMARY

In general, this disclosure describes techniques for generating predicted images indicating the future appearance of a wound. More specifically, this disclosure describes example techniques for generating a predicted image of the future appearance of a wound based on applying machine learning models to a time series of actual images of the wound. The predicted image can represent the probable appearance of the wound days or weeks in the future. This can enable a medical practitioner to make an early determination of the treatments or treatment parameters to be used to treat the wound based on a reasonably accurate prediction of the probable future appearance of the wound given a particular treatment or treatment parameters.

As described herein, a prediction system can receive image captures, a time series of images of a wound prior to treatment and/or during preliminary stages of treatment, and generate a predicted image of the wound as it would appear at a future point in time given a particular treatment or treatment parameters. A processing unit of the prediction device receives the image data, and provides the image data to a machine learning model that has been trained to generate the predicted image of the future appearance of the wound based on the image data, treatment method, and/or treatment parameters. In the various examples set forth herein, the prediction system can receive a time series of images from an initial period, for example, an initial image of the wound prior to treatment and images of the wound captured after treatment has commenced, and process the time series of images to generate a predicted image of the wound as it would likely appear after days or weeks of treatment.

Existing methods of wound treatment typically rely on the current state of the wound to guide treatment decisions. The techniques of this disclosure may provide at least one technical advantage over existing methods. For example, a practical application of the techniques disclosed herein is a prediction system that can generate a predicted image of the future appearance of a wound that can be used to guide decisions regarding the treatments and/or treatment parameters that will result in improved outcomes with respect to wound healing and wound appearance. As treatment progresses, a prediction system using the techniques disclosed herein can received further image captures of the wound, and can generate new predicted images of the future appearance of the wound. These new predicted images can be used to determine whether a current treatment plan is optimal or whether the treatment of the wound needs to be modified or replaced with a new treatment.

In one example, this disclosure describes a system that includes a memory; and a processing unit having one or more processors coupled to the memory, the one or more processors configured to execute instructions that cause the processing unit to: obtain image capture data for a sequence of one or more images representative of an appearance of a wound at a corresponding image capture time, each of the images prior to a final image of the sequence of images separated by a sampling time interval between the image and a next image, pass the image capture data for the sequence of images through a machine learning model trained to generate image data representing one or more predicted images of the future appearance of the wound, each of the one or more predicted images representative of a future appearance of the wound at a corresponding future time, the machine learning model trained using historical image data, the historical image data comprising one or more historical image data sets, each historical image data set of the one or more historical image data sets comprising image data for a historical sequence of images of an appearance of a corresponding historical wound, wherein a prediction time interval between the future time and a capture time of a last image of the sequence of images is greater than each of the sampling time intervals, and output the image data representing the one or more predicted images of the future appearance of the wound.

In another example, this disclosure describes a method that includes obtaining, by a processing unit comprising one or more processors, image capture data for a sequence of one or more images representative of an appearance of a wound at a corresponding image capture time, each of the images prior to a final image of the sequence of images separated by a sampling time interval between the image and a next image; passing the image capture data for the sequence of images through a machine learning model trained to generate image data representing one or more predicted images of the future appearance of the wound, each of the one or more predicted images representative of a future appearance of the wound at a corresponding future time, the machine learning model trained using historical image data, the historical image data comprising one or more historical image data sets, each historical image data set of the one or more historical image data sets comprising image data for a historical sequence of images of an appearance of a corresponding historical wound, wherein a prediction time interval between the future time and a capture time of a last image of the sequence of images is greater than each of the sampling time intervals; and outputting the image data representing the one or more predicted images of the future appearance of the wound.

In another example, this disclosure describes a method that includes receiving historical image data, the historical image data comprising a plurality of historical image data sets, each historical image data set of the historical image data sets comprising image data for a historical sequence of images of a corresponding wound, each image of the historical sequence of images prior to a final image of the historical sequence of images separated by a sampling time interval between the image and a next image; for each historical image data set of the plurality of historical image data sets, training the machine learning model to generate one or more predicted images of the future appearance of the wound, each image corresponding to a future time from the historical sequence of images, wherein a prediction time interval between the future time and a capture time of a last image of the historical sequence of images is greater than each of the sampling time intervals; and adjusting weights in layers of the machine learning model based on differences between the one or more predicted images and one or more target images associated with the wound.

In a further example, this disclosure describes a system that includes means for obtaining image capture data for a sequence of one or more images representative of an appearance of a wound at a corresponding image capture time, each of the images prior to a final image of the sequence of images separated by a sampling time interval between the image and a next image; means for passing the image capture data for the sequence of images through a machine learning model trained to generate image data representing one or more predicted images of the future appearance of the wound, each of the one or more predicted images representative of a future appearance of the wound at a corresponding future time, the machine learning model trained using historical image data, the historical image data comprising one or more historical image data sets, each historical image data set of the one or more historical image data sets comprising image data for a historical sequence of images of an appearance of a corresponding historical wound, wherein a prediction time interval between the future time and a capture time of a last image of the sequence of images is greater than each of the sampling time intervals; and means for outputting the image data representing the one or more predicted images of the future appearance of the wound.

In a still further example, this disclosure describes a system that includes means for receiving historical image data, the historical image data comprising a plurality of historical image data sets, each historical image data set of the historical image data sets comprising image data for a historical sequence of images of a corresponding wound, each image of the historical sequence of images prior to a final image of the historical sequence of images separated by a sampling time interval between the image and a next image; for each historical image data set of the plurality of historical image data sets, means for training the machine learning model to generate one or more predicted images of the future appearance of the wound, each image corresponding to a future time from the historical sequence of images, wherein a prediction time interval between the future time and a capture time of a last image of the historical sequence of images is greater than each of the sampling time intervals; and means for adjusting weights in layers of the machine learning model based on differences between the one or more predicted images and one or more target images associated with the wound.

The details of at least one example of the disclosure are set forth in the accompanying drawings and the description below. Other features, objects, and advantages of the disclosure will be apparent from the description and drawings, and from the claims.

### BRIEF DESCRIPTION OF DRAWINGS

FIGS. 1A-1C are block diagrams illustrating a system for generating a predicted image of the future appearance of a wound, in accordance with at least one example technique described in this disclosure.

FIGS. 2A and 2B are block diagrams illustrating a training system, in accordance with at least one example technique described in this disclosure.

FIG. 3 is a block diagram illustrating another training system, in accordance with at least one example technique described in this disclosure.

FIGS. 4A-4C are block diagrams illustrating example training frameworks for the training systems illustrated in FIGS. 2 and 3, in accordance with at least one example technique described in this disclosure.

FIG. 5 is a block diagram of an example processing unit of a system for generating a predicted image of the future appearance of a wound, in accordance with at least one example technique described in this disclosure.

FIG. 6 is a flowchart illustrating example operations of a method for generating a predicted image of the future appearance of a wound, in accordance with at least one example technique described in this disclosure.

FIG. 7 is a flow diagram illustrating an example operation of a training system, in accordance with one or more techniques of this disclosure.

### DETAILED DESCRIPTION

Systems and techniques are described for generating a predicted image of a future image of a wound based on current image captures of the wound and metadata associated with the image captures. A prediction system can receive image captures of a wound taken by a patient using the patient's own image capture device (e.g., smartphone camera, digital camera etc.) The image captures can be a time series of the samples over an initial sample period. The prediction system can generate a predicted image of the future appearance of the wound as it would appear at a future point in time, perhaps days or weeks in the future. The difference between the future point in time and the last sample of the time series may be referred to as a prediction interval. The sample period can be comparatively much shorter than the full treatment period of the wound, i.e., the sampling period may be much shorter than the prediction interval.

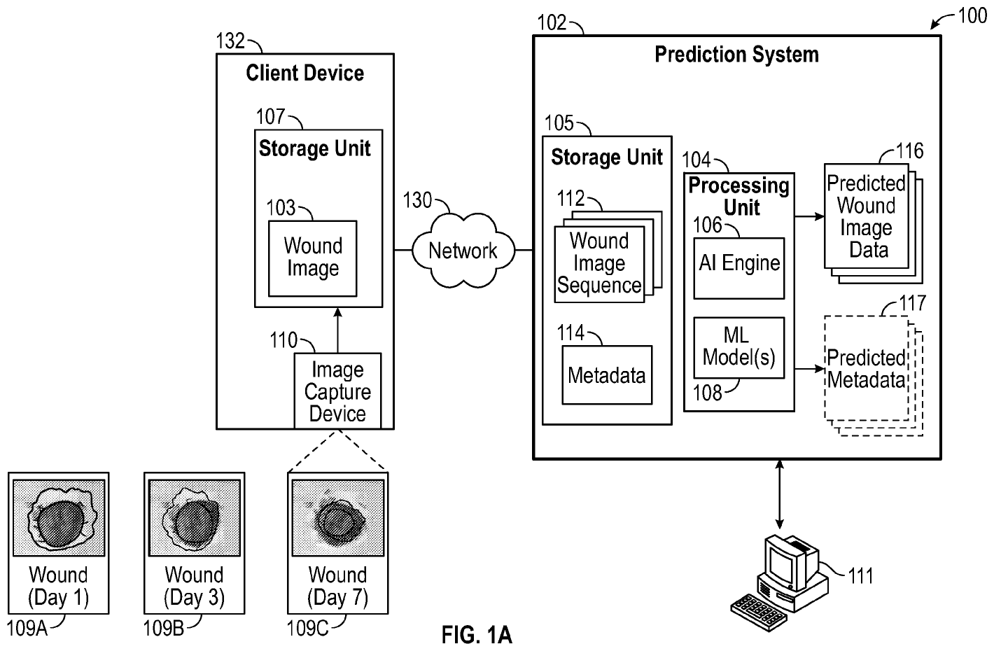

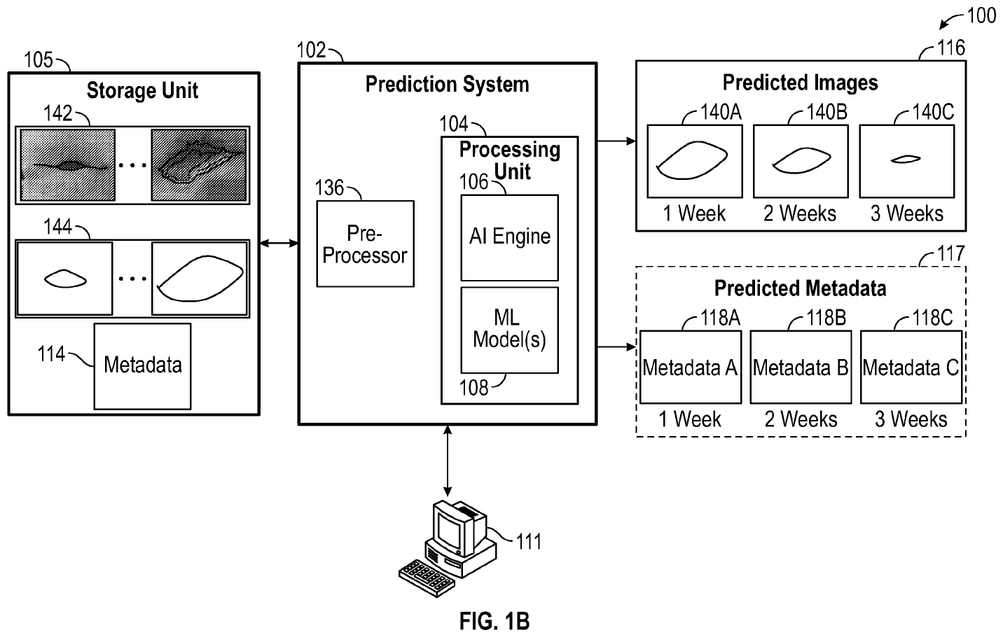

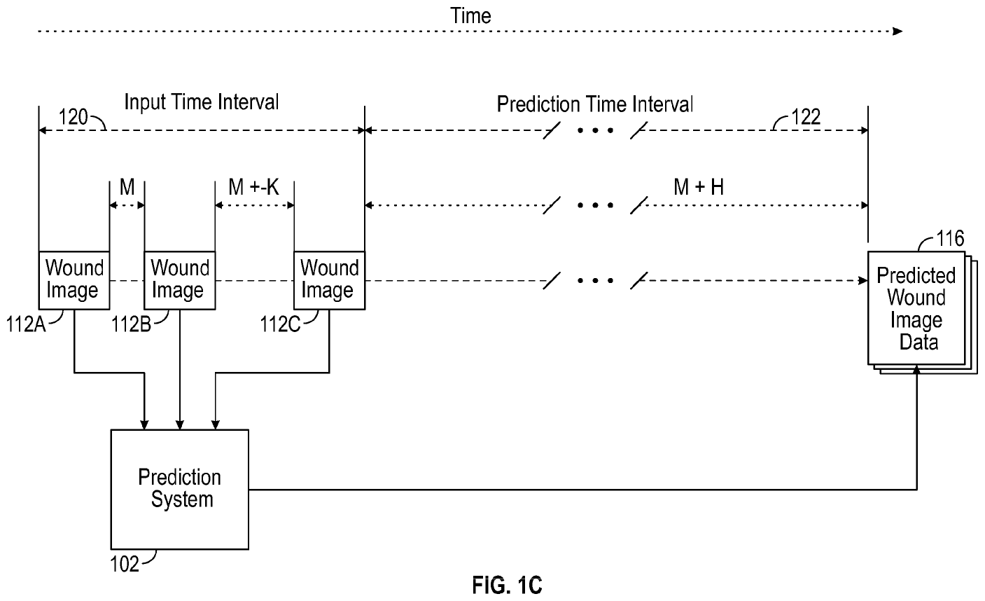

FIG. 1A is a block diagram illustrating a system for generating a predicted image of the future appearance of a wound, in accordance with at least one example technique described in this disclosure. In some aspects, system 100 includes prediction system 102 and a client device 132. Prediction system 102 and client device 132 may be communicatively coupled to one another via network 130. Network 130 may be any type of network, including a local area network, a wide area network, or a network that is part of the Internet.

Client device 132 may be any type of computing device having an image capture device 110. In some aspects, client device 132 may be a smartphone, for example, a smartphone belonging to a patient or other person associated with the patient. In some aspects, client device 132 may be a camera at a doctor's office, hospital, or other medical facility.

Image capture device 110 obtains one or more images 103 of a wound. In the example illustrated in FIG. 1A, image capture device 110 captures three images at different points in time after a patient is wounded. In this example, image capture device 110 captures an image of a wound 109A on the first day the patient is wounded, an image of the wound 109B three days after the patient is wounded, and an image of the wound 109C seven days after the patient is wounded.

Image capture device 110 may be a camera or other components configured to capture image data representative of a wound. Image capture device 110 may include components capable of capturing image data, such as a video recorder, an infrared camera, a CCD (Charge Coupled Device) array, or a laser scanner. Although one image capture device 110 is shown in FIG. 1A, there may be multiple image capture devices 110. Images 109 may all be captured from the same device, or they may be captured from different devices. For example, an image of wound 109A may be captured by a client device 132 at a medical facility treating or diagnosing the patient's wound, while images of wounds 109B and 109C may be captured by a patient owned client device 132.

In some aspects, an image 103 may be represented as a two-dimensional image. In some aspects, an image 103 can be a three-dimensional (3D) volume of images. For example, an image may be represented as a 3D volume of image data recorded over a relatively small period of time. As an example, the 3D volume may be a video recording. The three dimensions of the volume can be an x dimension, a y dimension, and a time dimension. Thus, capturing image data can refer to capturing a 2D image or recording multiple frames of image data as a 3D volume over a time period.

Image capture device 110 may store captured images 103 on storage unit 107 of client device 132. Client device 132 may transmit captured images 103 to prediction system 102 via network 130. In some aspects, client device 132 may transmit a captured image 103 to prediction system 102 individually, i.e., before another image of the wound 109 is captured. In some aspects, client device 132 may store multiple images 103 of the wound on storage unit 107, and transmit the multiple images together to prediction system 102.

Prediction system 102 can receive images 103 transmitted by client device 132 and store the received images in storage unit 105 as part of wound image sequence 112. In some aspects, wound image sequence may be a single image of a wound 109. In some aspects, wound image sequence 112 may be a time series of images of a wound 109 (e.g., images captured of wound 109A-109C over a period of time). Prediction system 102 may store one or more timestamps as part of metadata 114 associated with wound sequence 112. The timestamps may be timestamps obtained from image data 103 indicating when the image was captured. A timestamp may be generated by prediction system indicating when the image was received. The timestamp may be stored with the image, or it may be stored as metadata 114. Metadata 114 may also include data such as the type of treatment being used to treat the wound (e.g., wound closure and healing treatments), products used to clean the skin and/or wound, and products used to dress the wound. As an example, metadata 114 may include one or more parameters that control a Negative-Pressure Wound Therapy (NPWT) system. A NPWT system can be configured to control fluid at a wound site based on one or more input parameters that regulate wound irrigation and/or instillation. Details on a NPWT system may be found in U.S. Provisional Patent Application No. 63/201,319 filed on Apr. 23, 2021, and entitled “Wound Therapy System.” the entirety of which is incorporated by reference herein.

Metadata 114 may also include characteristics of the wound itself, such as the size of the wound, location of the wound, tissue type affected, signs and/or symptoms of infection and exudate (secretions) associated with the wound or other data associated with the wound. Metadata 114 may also include demographic information regarding the patient (age, gender, ethnicity, etc.), or other data associated with the patient. For example, metadata 114 may include data from a patient record such as weight, body mass index, and the like, personal and/or family medical histories and co-morbidities, past and current diagnoses, e.g., diabetes, obesity, cardiovascular disease, cholesterol, blood pressure, and the like, prescribed medications including dosage and frequency of use, blood lab results and values, genetic test results, allergies and allergy test results, and any other suitable patient health record data.

Processing unit 104 of prediction system 102 can read and process wound image sequence 112. For example, prediction system 102 may read and process wound sequence 112 in response to receiving a command from a user via user interface 111. Processing unit 104 can utilize artificial intelligence (AI) engine 106 and machine learning model 108 to process the image data of wound image sequence 112, and optionally, metadata 114, to generate predicted wound image data 116, and optionally, predicted metadata 117. In some aspects, AI engine 106 and machine learning model 108 may implement a neural network. For example, machine learning model 108 can define layers of a neural network that has been trained using techniques described herein to receive wound image sequence 112 as input and to generate predicted wound image data 116 as output. In some aspects, predicted wound image data 116 is in the same form as image data for images in wound image sequence 112. For example, if the images in wound image sequence 112 are 2D images, then predicted wound image data 116 can represent a 2D image. Similarly, if the images in wound image sequence 112 are 3D volumes, then predicted wound image data 116 represents a 3D volume. In some aspects, predicted wound image data 116 can have a different form from the image data for images in wound image sequence 112. For example, the images in wound image sequence 112 can be 3D volumes. Prediction system 102 can generate predicted wound image data 116 as a 2D image.

In some aspects, prediction system may extract image processing features (e.g., difference from the initial image over time, gradient based images etc.) and use such features as an additional input to prediction system 102 and machine learning model 108.

In some aspects, predicted wound image data 116 can be data for a single predicted wound image. In some aspects, predicted wound image data 116 can be a sequence of images (e.g., 2D images or 3D volumes). In some aspects, the sequence of images may be a time sequence of predicted images having a temporal order. For example, the first image of predicted wound image data 116 may be an earliest predicted image, and the last image may be a predicted image at a furthest point in time of the sequence.

In the example shown in FIG. 1A, wound image sequence 112 is presumed to be a sequence of multiple images. In some aspects, wound image sequence 112 may be a single image (e.g., a single 2D image or single 3D volume), and machine learning model 108 may be trained to generate predicted wound image data 116 from a single input image.

In some examples, user interface 111 allows a user to control system 100. User interface 111 can include any combination of a display screen, a touchscreen, buttons, audio inputs, or audio outputs. In some examples, user interface 111 is configured to power on or power off any combination of the elements of system 100, provide configuration information and other input for prediction system 102 and/or processing unit 104, and display output from prediction system 102.

FIG. 1B is a block diagram illustrating another system for generating a predicted image of the future appearance of a wound, in accordance with at least one example technique described in this disclosure. In the example illustrated in FIG. 1B, prediction system 102 includes preprocessor 136 that can process wound images 103 prior to prediction system 102 generating predicted wound image data 116. As noted above, wound images 103 may be generated using an image capture device 110 of client device 132. In some aspects, client device 132 may be a smartphone or other handheld device. Images of a wound captured over time may be captured under different conditions. For example, images of a wound may be captured at different distances, different angles, and different lighting conditions. The images may also have varying amounts of background elements included in the image. These images in their form prior to preprocessing are referred to as unregistered wound image sequence 142. Preprocessor 136 can use image segmentation techniques to segment the wound from the image data to exclude non-wound elements such as background elements and/or nonaffected body portions. Preprocessor 136 can then align the segmented wound images with respect to scale and angle. The segmented and aligned wound images may be referred to as registered wound image sequence 144. In some aspects, processing unit 132 can uses registered wound image sequence 142 as input to generate predicted wound image data 116. In the example shown in FIG. 1B, prediction system 132 generates three predicted images of the future appearance of a wound 140A-140C representing the appearance of the wound one week in the future (140A), two weeks in the future (140B), and three weeks in the future (140C).

In some aspects, prediction system 102 may generate predicted metadata 117 in addition to, or instead of, predicted images 116. Predicted metadata 117 can include predicted future wound properties such as predicted wound geometry (e.g., wound area, wound depth, wound positioning), wound healing stage etc. In the example shown in FIG. 1B, prediction system 132 generates three sets of predicted metadata associated with the wound, metadata 118A-118C. These sets of metadata represent wound properties at one week in the future (metadata 118A), two weeks in the future (metadata 118B), and three weeks in the future (metadata 118C).

FIG. 1C is a block diagram illustrating input image data for a system for generating a predicted image of the future appearance of a wound, in accordance with at least one example technique described in this disclosure. In the example illustrated in FIG. 1C, image capture device 110 has captured image data for images 112A-112C of an example wound sequence 112 at various points in time over input time interval 120. In this example, the time interval between the image captures of image 112A and image 112B can is m days. The time interval between the image captures of image 112B and image 112C is m±k days. The prediction time interval 122 between the time image 112C is captured and generation of predicted wound image data 116 is m+h days. The prediction time interval 122 does not represent an amount of actual time passing. Instead, the prediction time interval m+h represents a simulated time interval between image 112C and predicted wound image data 116. The actual time interval between image 112C and the generation of predicted wound image data 116 can be merely the amount of time it takes prediction system 102 to generate predicted wound image data 116. Prediction time interval 122 can be much longer than input time interval 120. For example, prediction time interval 122 can be longer than twice input time interval 120, and in some examples, can be even much longer, e.g., weeks longer. However, prediction time interval 122 is not limited in this respect, and, in some cases, may be equal or nearly equal to an input time interval.

As an example, image capture device 110 may create wound image sequence 112 by capturing images 103 of a wound 109 (FIG. 1A) every other day for a five day period. Prediction system 102 can process wound image sequence 112 to generate predicted wound image data 116, representing a predicted image of the future appearance of a wound at a future point in time, for example, two weeks in the future. The predicted wound image data 116 can be used to determine whether the current treatment plan for the wound is acceptable to the doctor and/or patient, or if a different or modified treatment plan should be considered. Using the techniques described herein, a user (or a user system) can use the predicted image of the future appearance of the wound to reach a conclusion regarding wound treatment much earlier than would be possible using currently existing methods. In the example described above, the user can reach a conclusion regarding wound treatment days or weeks earlier than current methods.

The example shown in FIG. 1C illustrates several aspects of wound image sequence 112 and predicted wound image data 116. A first aspect is that there may be long sampling intervals. Each input image may be days apart from each other. Within these intervals, there can be many changes governed by potentially non-linear processes involved in wound healing. Thus, it is challenging to produce an accurate future image or 3D volume of a wound.

A second aspect is that the time intervals between image captures can be inconsistent and non-uniform. As shown in FIG. 1C, the first two samples could be m days apart whereas the time between the next two samples can be more or less than m days (i.e., m±k days) apart. It is also possible that an expected interval may be relatively large because of missing or corrupted data. Thus, k could be even higher than it would be when the wound image capture is missing or corrupted.

A third aspect is that the prediction time interval (e.g., m+h) associated with predicted wound image data 116 can be very long compared to the intervals between image captures of a wound. For example, the prediction time interval 122 between a last captured image of wound image sequence 112 (e.g., image 112C) and predicted wound image data 116 may be days to weeks apart.

A user such as a clinician or medical practitioner, can operate prediction system 102 to determine the effect that different treatments or different treatment parameters (e.g., NPWT parameters) will likely have on wound healing and the predicted future appearance of the wound. For example, a set of one or more current images of a wound along with metadata describing the wound may be provided to prediction system 102. A user may provide further metadata, such as data describing a treatment method, or parameters of wound treatment system as input to prediction system 102. Prediction system 102 can generate a predicted future image of the wound based on the current images of the wound and the input metadata. The user can vary different input parameters such as the proposed treatment and/or treatment system parameters, and can select for application to the wound the treatment and/or treatment parameters that produce a desired result with respect to the predicted image of the future appearance of the wound.

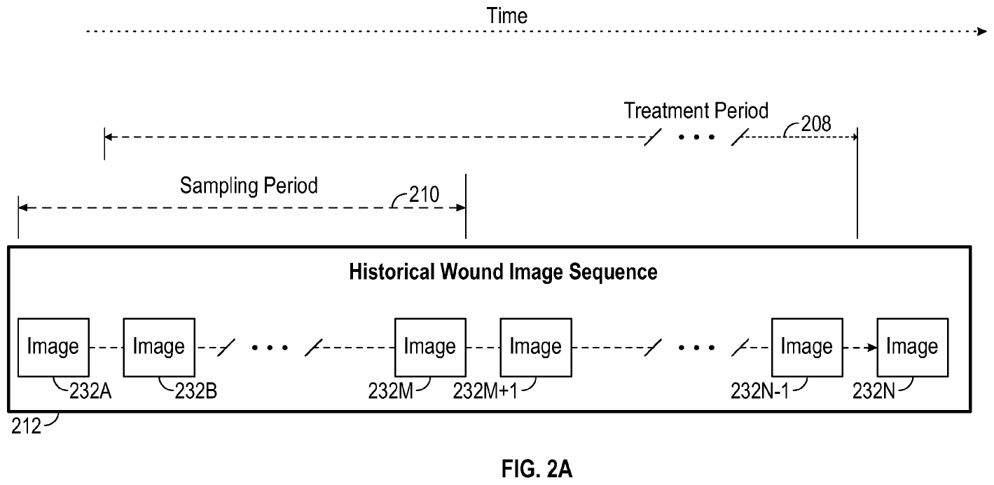

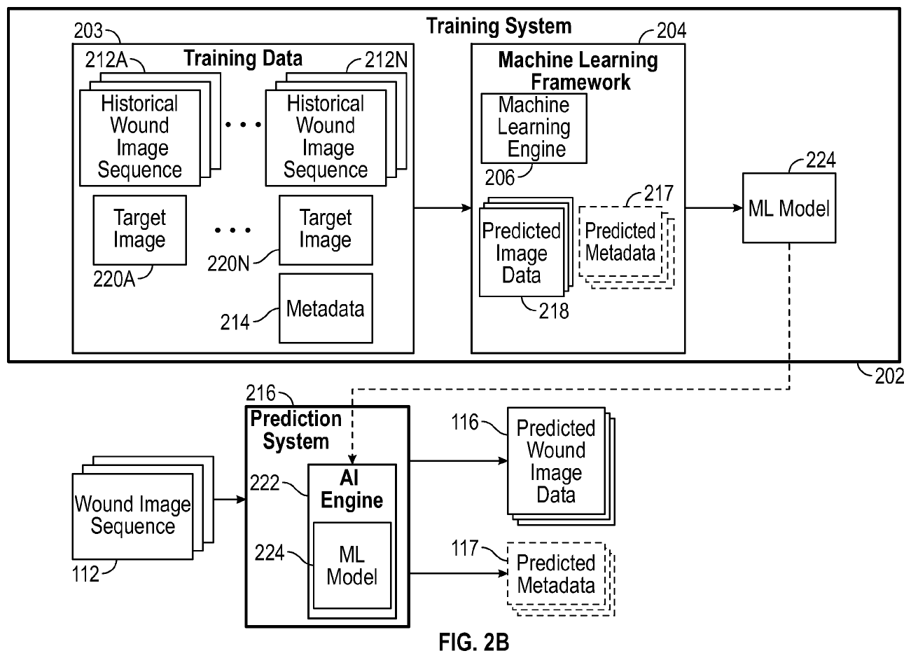

FIG. 2A is a block diagram illustrating training data for a training system such as the training system discussed below with reference to FIG. 2B. In some aspects, the training data includes multiple historical wound image sequences. A historical wound image sequence 212 includes images 232A-232N that comprise image data for a sequence of images of a corresponding wound captured at different points in time. Images 232A-232M of historical wound image sequence can be images captured during sampling period 210. Sampling period 210 can include images captured prior to the completion of treatment of the wound, which may include images captured days or weeks prior to the anticipated completion of the treatment. As an example, image 232A may be captured prior to the initiation of treatment when a patient first visits a medical practitioner to seek treatment of their wound. Generally speaking, images 232B-232M may be captured at any point prior to completion of the treatment, for example, during early stages of treatment of the wound. Images 232M+1-232N can be images captured during later stages of the treatment period 208 of the wound. The final image of the sequence, image 232N, can be a target image for the sequence. That is, the final image 232N may be used as the ground truth with respect to the appearance of a corresponding wound subject to a given treatment at a desired point in the treatment of the wound, for example, at the end of treatment or a point in time after treatment has been completed.

FIG. 2B is a block diagram illustrating a training system, in accordance with at least one example technique described in this disclosure. Training system 202 can include a machine learning framework 204 that includes machine learning engine 206. Machine learning framework 204 can receive training data 203, and process the training data to generate machine learning model 224. In some aspects, machine learning framework 204 includes machine learning engine 206 that may use supervised or unsupervised machine learning techniques to train machine learning model 224. In some aspects, machine learning engine 206 can be a deep learning engine implementing a convolutional neural network (CNN). In some aspects, machine learning engine 206 can be a generative adversarial network (GAN), for example. As an example, machine learning engine can be a T-Adversarial GAN. In some aspects, machine learning engine 206 can be a U-Net based machine learning engine, including U-Net 2D and U-Net 3D architectures. U-Net architectures can be used to preserve content such as spatial information in the training data. U-Net architectures typically have contracting and expansive paths, and in conjunction with skip connection in the layers, can be used to link corresponding feature maps on the encoder and decoder. The linking of feature maps can facilitate reuse of features in the encoder, thereby reducing information loss. Additionally, U-Net architectures can be computationally efficient and can be trained with a relatively small dataset.

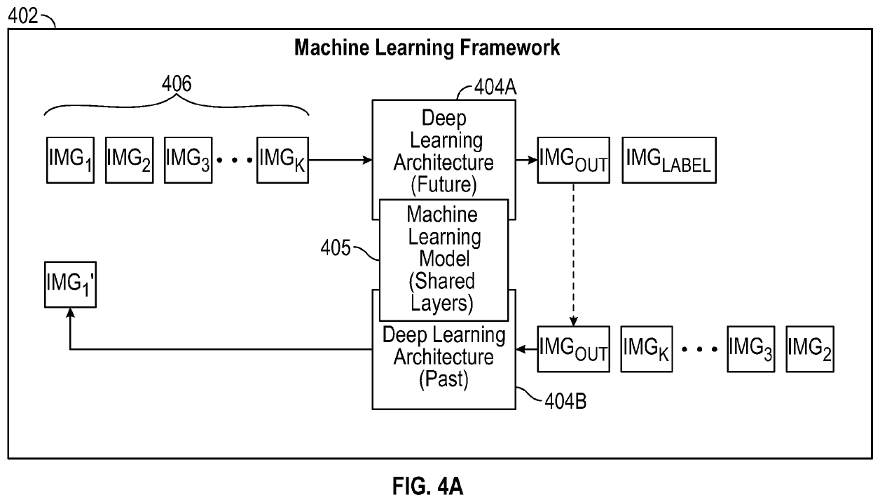

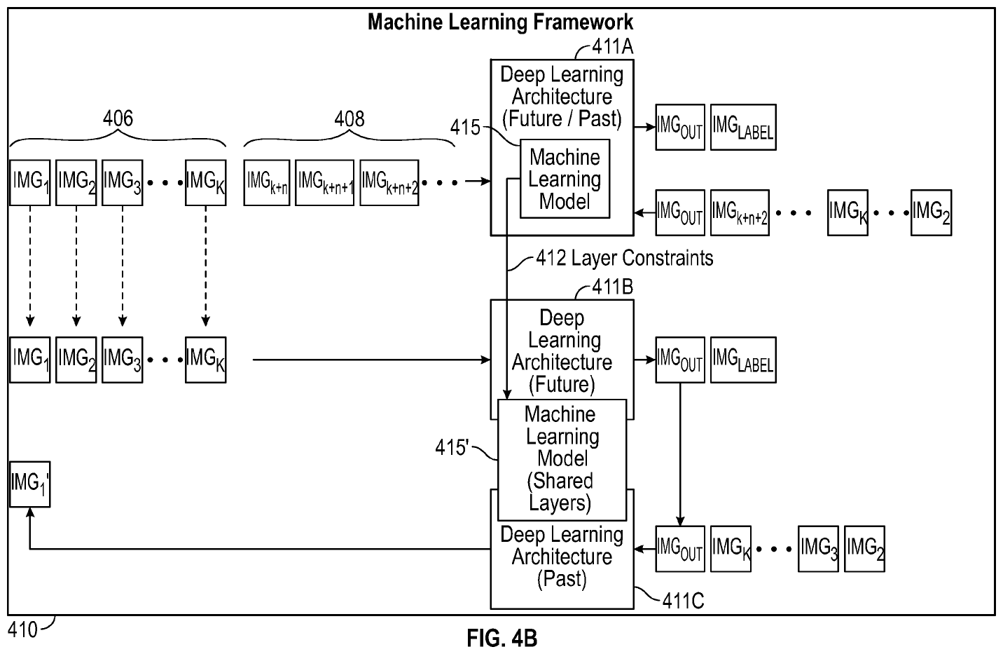

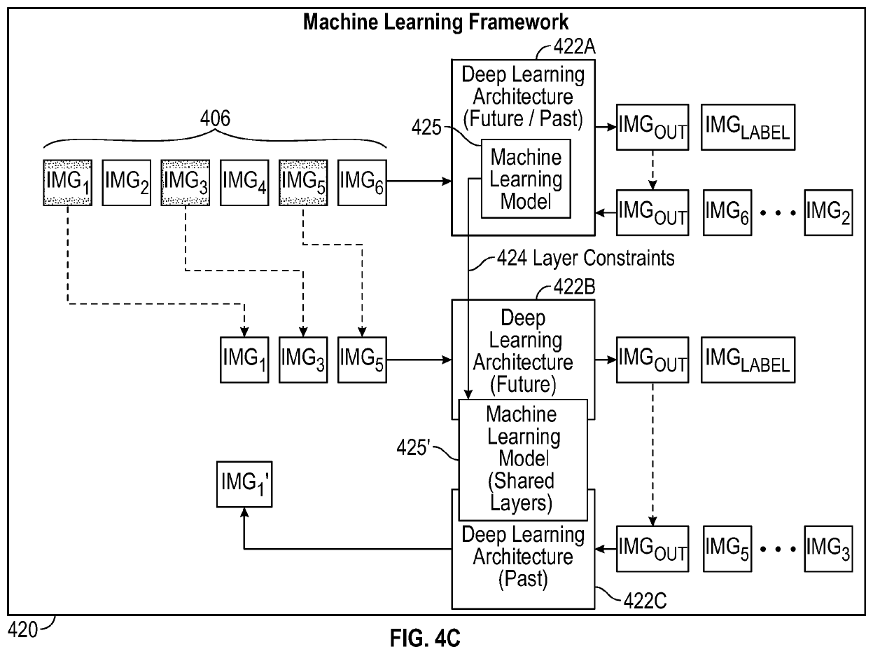

In some aspects, machine learning framework 204 may implement multiple machine learning techniques that can be applied together when training machine learning model 224. For example, machine learning engine 206 may be a U-Net engine and machine learning framework may apply cyclic learning techniques using machine learning engine 206. Further details on machine learning framework and cyclic learning are provided below with respect to FIGS. 4A-4C.

Training data 203 can include historical wound image sequences 212A-212N (generically referred to as a historical wound image sequence 212). Each historical wound image sequence 212 in the training data is a time sequence of images of a particular wound captured or recorded over a time period prior to training machine learning model 224. For example, historical wound image sequence 212A may be image data for a sequence of images showing the appearance of a first wound over time, historical wound image sequence 212B may be image data for a sequence of images showing the appearance of a second wound over time, historical wound image sequence 212C may be image data for a sequence of images showing the appearance of a third wound over time, etc.

Each historical wound image sequence 212A-212N in training data 203 can have a corresponding target image 220A-220N. The target image for an image sequence is the “ground truth” final image e.g., an actual image of the wound associated with the image sequence captured at the end of the treatment period.

Training data 203 may also include metadata 214 that can be used for training machine learning model 224. Metadata 214 can include timestamps indicating when images in historical wound image sequence 212 were captured. Metadata 214 may include patient demographic information, for example, data from a patient record such as weight, body mass index, and the like, personal and/or family medical histories and co-morbidities, past and current diagnoses, e.g., diabetes, obesity, cardiovascular disease, cholesterol, blood pressure, and the like, prescribed medications including dosage and frequency of use, blood lab results and values, genetic test results, allergies and allergy test results, and any other suitable patient health record data. Metadata 214 may include wound information such as wound geometry (e.g., wound location, wound depth, wound size, etc.), affected tissue type, signs, or symptoms of infection, and/or healing stage. Metadata 214 may also include data such as the type of treatment that was used to treat the wound (e.g., wound closure and healing treatments), products that were used to clean the skin and/or wound, and products that were used to dress the wound. As an example, metadata 214 may include one or more parameters values of a NPWT system used to treat the wound. Metadata 214 may also include characteristics of the wound itself, such as the size of the wound, location of the wound, tissue type affected, signs and/or symptoms of infection and exudate (secretions) associated with the wound at the time the historical image was captured. Metadata 214 may also include demographic information regarding the patient (age, gender, etc.), or other data associated with the patient or wound at the time the historical image was captured. In some aspects, metadata 214 may be added to training system 202 (or prediction system 102 of FIG. 1) by padding the metadata information on the boundary of the image. In some aspects, metadata 214 may be added as a vector to a latent feature vector produced in a mid-layer of the machine learning model 224.

Training system 202 provides training data 203 to machine learning framework 204 for processing by machine learning engine 206. Machine learning engine 206 processes historical wound image sequence 212 to generate a predicted image data 218. Predicted image data 218 can include a sequence of predicted images of the future appearance of a wound that each have an associated future time. Machine learning framework 204 can compare the predicted image to target image 220 associated with historical wound image sequence 212 to determine differences between the predicted image and target image 220. The difference between predicted image and target image 220 is used to update training weights in machine learning model 224 to attempt to improve the model's ability to generate accurate predicted images of the future appearance of a wound. In some aspects, the weights in machine learning model 224 can be adjusted using a loss function, such as reconstruction loss or GAN loss.

In some aspects, machine learning framework 204 may also train machine learning model 224 to generate predicted metadata 217 using historical metadata information (e.g., metadata 214) associated with historical wound image sequence 212. Metadata 214 may be a historical sequence of metadata and target metadata that corresponds to historical wound image sequence 212 and target images 220. Machine learning framework can compare the predicted metadata 217 with the target metadata and adjust the machine learning model based on differences between the predicted metadata 217 and the target metadata.

After training system 202 has trained machine learning model 224, the model may be deployed to prediction system 216. Prediction system 216 may be an implementation of prediction system 102 of FIG. 1. Prediction system 216 can receive wound image sequence 112 and process the image sequence using AI engine 222 and the deployed machine learning model 224 to generate predicted wound image data 116, and/or predicted metadata 117.

As shown in FIG. 2B, machine learning framework 204 can generate predicted image data 218 that can include a sequence of images (e.g., 2D images or 3D volumes). In some aspects, machine learning framework 204 can generate predicted image data 218 that can be a single 2D image or 3D volume. Additionally, a historical wound image sequence 212 can be a sequence of multiple images as shown in FIG. 2B. In some aspects, a wound image sequence 212 may be a single image (e.g., a single 2D image or single 3D volume), and machine learning framework 204 may train machine learning model 224 to generate predicted image data 218 from a single input image.

In some aspects, machine learning engine 206 can implement a weighted loss function that assigns different weights to images in predicted image data 218. For example, the weighted loss function may assign a greater weight to an image that is later in the sequence of images that an image that is earlier in the sequence. In other words, a first predicted future image having an associated predicted future time that is earlier than the predicted future time associated with a second predicted future image will have a weight that is less than the second predicted future image. This can be beneficial because a predicted future image that is accurate and later in time in the sequence of predicted images can be more valuable to an end user than another predicted image that is predicted for a future time that is earlier in the sequence. In some examples, these weights could also be learned from data. For example, the machine learning model can automatically learn the relevance and importance of each image data in the input.

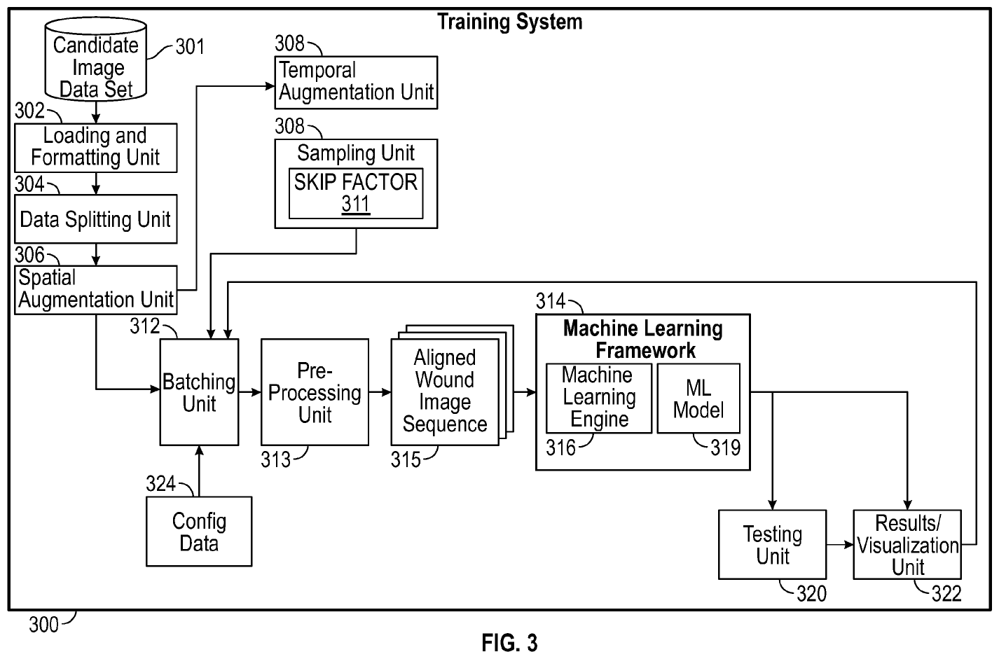

FIG. 3 is a block diagram illustrating further aspects of a training system, in accordance with at least one example technique described in this disclosure. In the example shown in FIG. 3, training system 300 includes loading and formatting unit 302, data splitting unit 304, spatial augmentation unit 306, temporal augmentation unit 308, sampling unit 310, batching unit 312, pre-processing unit 313, machine learning framework 314, testing unit 320, and results visualization unit 322, loading and formatting unit 302, data splitting unit 304, spatial augmentation unit 306, temporal augmentation unit 308, sampling unit 310, batching unit 312, pre-processing unit 315, machine learning framework 314, testing unit 320, and results visualization unit 322 can be implemented as a configurable pipeline to process candidate image data set 301 into batches of image data sets to be used by machine learning framework 314 to train machine learning model 319.

Loading and formatting unit 302 can process a candidate image data set 301 to format image sequences in candidate image data set 301 into a form that the training system can process. For example, images may be scaled, resized, cropped etc. so that they are in a format that is compatible with machine learning framework 314.

Data splitting unit 304 can divide candidate image data set 301 into training data, testing data, and/or validation data. For example, input parameters may specify percentages of a data set to use as training data, testing data, and/or validation data.

Spatial augmentation unit 306 can increase the amount of training data by transforming an existing image into one or more additional training images. For example, an image may be transformed by taking a section of the image and moving the section left, right, along a diagonal axis, rotating the image, mirroring the image etc. to create a new image that can be included in the training data.

Temporal augmentation unit 308 can control the selection of images from candidate image data set 301 based on temporal aspects of the candidate training data. Temporal augmentation unit 308 can select image sequences based on where the image is positioned on a time axis. As an example, temporal augmentation unit 308 can select images based on a starting time and an ending time.

Sampling unit 310 can select images from the training data according to a skip factor 311. For example, rather than including every image in candidate image data set 301, sampling unit 310 may select a subset of images in the candidate data set. Skip factor 311 may be used to control the manner in which images are selected. For example, a skip factor of four may cause the sampling unit 310 to skip four images of the candidate data set before selecting a next image for inclusion in training data.

Configuration data 324 can include data that determines data sources, hyperparameters, machine learning parameters, types of machine learning etc. for use by machine learning framework 314.

Batching unit 312 creates and controls batches of training data that are to be processed as a unit. For example, a first batch of training data may be used to train a first machine learning model 319 and a second batch of training data may be used to train a second machine learning model 319. Batching unit 312 may use configuration data 324 to determine which data sources to use for a batch of training data. Batching unit 312 may also use configuration data 324 to specify configuration parameters that machine learning framework 314 is to use when training machine learning model 319 using the corresponding batch of training data.

Batching unit 312 can provide a batch of training data to machine learning framework 314 for use in training machine learning model 319. In some aspects, batching unit 312 can provide the training data to pre-processing unit 313. Pre-processing unit 313 can apply image segmentation techniques to each wound image in the training set of images to segment the wound from other image data to exclude non-wound elements such as background elements and/or nonaffected body portions. Preprocessor 136 can then align the segmented wound images with respect to scale and angle. The segmented and aligned wound images may be referred to as aligned wound image sequence 315.

Machine learning framework 314 can include machine learning engine 316. In some aspects, machine learning framework 314 and/or machine learning engine 316 can be implementations of machine learning framework 204 and/or machine learning engine 206 of FIG. 2B. As discussed above, machine learning engine 316 can be a deep learning engine implementing a CNN, a GAN, a U-Net based machine learning engine, including U-Net 2D and U-Net 3D architectures. Machine learning framework 314 can train machine learning model 319 using the techniques described herein to generate a predicted image of the future appearance of a wound.

Testing unit 320 can test machine learning model 319 to determine the accuracy of predicted future wound images generated using machine learning model 319. As described above, candidate image data set 301 can be split into training data and testing data. Machine learning model 319 is trained using the training data. Testing unit 320 can receive historical wound image sequences in the testing data as input and can generate, using machine learning model 319, predicted wound images as output.

As an example, the test data may include a historical sequence of images of a wound, with a first portion of the images in the sequence being captured during the sampling period 210, and other images in the sequence captured after sampling period 210. The last image in the sequence can be the target wound image. Testing unit 320 may apply machine learning model 319 to the first portion of the historical sequence of images to generate a predicted wound image. Testing unit 320 can then compare the predicted wound image with the target wound image and determine, based on the comparison, the accuracy of the predicted wound image. Testing unit 320 can determine various measurements of the performance of machine learning model 319, and compare the measurements with other machine learning models that may have been generated using different training parameters and/or training data. The results of the comparison can be used to determine a machine learning model 319 that produces better (e.g., more accurate) predicted wound images.

Results visualization unit 322 can provide feedback to a user regarding the training of machine learning model 319. For example, results visualization unit 322 can output statistics regarding the accuracy of predicted images generated by machine learning model 319. In some aspects, results visualization unit 322 can output examples of input wound image sequences and the predicted wound image generated by machine learning model 319. A user can utilize the output of results visualization unit 322 to determine if any adjustments need to be made with respect to training machine learning model 319. For example, a user may adjust hyperparameters, prediction time intervals, or other configuration data 324 and signal batching unit 312 to begin to provide another batch of training data to train a new machine learning model 319. Results visualization unit 322 can provide output that can be used to compare the performance of machine learning model 319 with other machine learning models.

Training system 300 need not include all of the components illustrated in FIG. 3, and in various implementations, training system 300 can include various combinations of one or more of the components illustrated in FIG. 3.

FIGS. 4A-4C are block diagrams illustrating example bi-directional machine learning frameworks used in the training systems illustrated in FIGS. 2 and 3, in accordance with at least one example technique described in this disclosure. In the examples shown in FIGS. 4A-4C, the machine learning model is to be trained to generate a predicted image of the future appearance of a wound using a sequence 406 of input wound images. In the examples illustrated in FIGS. 4A-4C, image sequence 406 is a sequence of k images IMG₁-IMGₖ, where IMG₁ is the first image in the sequence and IMGₖ is the last image in the sequence. Input image sequence 406 can be images of a wound captured during a sampling period. IMGₒᵤₜ is a predicted future image generated by the machine learning framework using image data selected from images 406. IMG_{label} is an image that was captured at the end of treatment or after treatment of the wound. IMG_{label} may be an image captured at a point days, or even weeks after input images 406 were captured. IMG_{label} represents a “ground truth” image, also referred to as a target image.

FIG. 4A is a block diagram illustrating a machine learning framework 402 that performs two passes through an image sequence to train a machine learning model to predict a future image of the appearance of a wound. Machine learning framework 402 can be an implementation of machine learning framework 204 of FIG. 2B and/or machine learning framework 314 of FIG. 3. Machine learning framework 402 includes two deep learning architectures 404A and 404B. Deep learning architecture 404A is used to train machine learning model 405 to generate a predicted future image from a sequence of past images, and deep learning architecture 404B is used to train machine learning model 405 to reconstruct a past image from later images and the predicted future image. Deep learning architecture 404A and 404B each may be a CNN (including U-Net 2D and U-Net 3D), a GAN, a T-Adversarial GAN, a Time Cyclic GAN, or a GAN using privileged information. In some aspects, deep learning architecture 404A and 404B share the layers in machine learning model 405. The shared layers provide a constraint on the learning where past information is linked with future information such that the predicted future image can be used to reconstruct a past image. In the example illustrated in FIG. 4A, the goal of the first pass is to generate, using deep learning architecture 404A, a predicted future image IMGₒᵤₜ that is the same or similar to the ground truth image IMG_{label}. IMGₒᵤₜ is compared with IMG_{label}, and the difference between IMGₒᵤₜ and IMG_{label} is used to update training weights in deep learning architecture 404A to attempt to improve the generated predicted future image IMGₒᵤₜ. In some aspects, the weights in machine learning model 405 can be adjusted using a loss function, such as reconstruction loss or GAN loss. The above-described process is generally the same as that used for single-direction training of some implementations.

In the second pass, a side goal is to use deep learning architecture 404B to generate a reconstructed first image in the sequence, IMG₁′ that is the same as or similar to the actual first image in the sequence, IMG₁, using subsequent images IMG₂-IMGₖ and the predicted future image IMGₒᵤₜ as input to deep learning architecture 404B. IMG₁′ is compared to IMG₁ (which is now considered the target image) and the difference is used to adjust weights in the layers of machine learning model 405. This second pass can make the layer weights more robust, and can avoid over-fitting the machine learning model to the training data.

FIG. 4B is a block diagram illustrating a machine learning framework 410 that performs two stages of bi-directional passes to train a machine learning model to predict a future image of the appearance of a wound. Machine learning framework 410 includes deep learning architectures 411A, 411B, and 411C (collectively “deep learning architectures 411”). Deep learning architectures 411 each may be a CNN (including U-Net 2D and U-Net 3D), a GAN, a T-Adversarial GAN, a Time Cyclic GAN, or a GAN using privileged information.

In some aspects, deep learning architecture 411A is implemented similarly to deep learning architectures 404A and 404B described above with reference to FIG. 4A. That is, deep learning architecture 411A can be bi-directional and can perform two passes through an image sequence, namely, a first pass the generates a predicted future image IMGₒᵤₜ from initial images in an image sequence 406, and a second pass that generates a reconstructed first image IMG₁ based on the predicted future image IMGₒᵤₜ and images subsequent to the first image in the image sequence. In some aspects, deep learning architecture 411A differs from deep learning architectures 404A and 404B of FIG. 4A by including a longer time range of image data in training a machine learning model. For instance, in addition to input images 406 collected prior to treatment of a wound and images at initial stages of a treatment, deep learning architecture 411A may also include images 408 collected during later stages of the treatment of the wound.

In the example illustrated in FIG. 4B, a first stage of generating machine learning model 415 includes using machine learning architecture 411A to train machine learning model 415 to generate a predicted future image IMGₒᵤₜ that is the same as or similar to the ground truth image IMG_{label}. Machine learning framework 410 compares IMGₒᵤₜ with IMG_{label}, and the difference between IMGₒᵤₜ and IMG_{label} is used to update training weights in machine learning model 415 to attempt to improve the generated predicted future image IMGₒᵤₜ. In some aspects, the weights in machine learning model 415 can be adjusted using a loss function, such as reconstruction loss or GAN loss. Additionally, deep learning architecture 411A trains machine learning model 415 to reconstruct the first image IMG₁ from IMGₒᵤₜ and images subsequent to IMG₁. As noted above, in the example illustrated in FIG. 4B, the input to deep learning architecture 411A during the first stage of machine learning can include both images captured prior to treatment (e.g., IMG₁-IMGₖ), and images captured after treatment has commenced, but prior to completion of treatment (e.g., images IMGₖ₊ₙ, IMGₖ₊ₙ₊₁, IMGₖ₊ₙ₊₂ etc.). Thus, in the first stage of training, machine learning framework 410 takes advantage of a longer time frame of data to improve the accuracy of machine learning model 415.

While it can be advantageous to use a longer time frame to improve the accuracy of a predicted future image, an aspect of the techniques disclosed herein is a machine learning model that can generate a predicted future image using images captured prior to treatment of a wound and images captured during earlier stages of the treatment of the wound without relying on images captured during later stages of treatment of the wound. Thus, in the example illustrated in FIG. 4B, during a second stage of machine learning, machine learning framework 410 continues to train machine learning model 415′ using images 406 captured prior to treatment of the wound and during the early stages of the treatment. As was the case with the first stage, the second stage of training can be bi-directional with deep learning architecture 411B sharing layers of machine learning model 415′ with deep learning architecture 411C. For example, deep learning architecture 411B trains machine learning model 415′ to generate a predicted future image IMGₒᵤₜ using input images 406 (e.g., IMG₁-IMGₖ) and compares IMGₒᵤₜ to IMG_{label} to determine adjustments to the weights of machine learning model 415′. Additionally, deep learning architecture 411C trains machine learning model 415′ to generate a reconstructed image IMG₁′ using IMGₒᵤₜ and IMGₖ-IMG₂ as input and compares IMG₁′ to IMG₁ to determine adjustments to the weights of machine learning model 415′.

Machine learning framework 410 can impose constraints 412 on the training of machine learning model 415′. For example, machine learning framework 410 can enforce a constraint that certain layers of machine learning model 415′ match the weights of corresponding layers of machine learning model 415. In some aspects, the constraint can be that the weights of the final layer of machine learning model 415′ match the weights of the final layer of machine learning model 415. In some aspects, the constraint can be that the weights of a middle layer of machine learning model 415′ match the weights of a corresponding middle layer of machine learning model 415.

In addition to the aspects discussed above, a further aspect of the disclosure illustrated in FIG. 4B is that additional data obtained in the future can be used to train machine learning model 415′. For example, machine learning model 415 may be trained using an initial set of training data. As further data becomes available at a future point in time, machine learning model 415′ may be trained as discussed above to potentially improve the accuracy of the predicted images.

In the example illustrated in FIG. 4B, machine learning framework 410 implements bi-directional training (i.e., cyclic training) to train both machine learning model 415 and machine learning model 415′. However, bi-directional training is not a requirement, and in some aspects, machine learning framework 410 can train either or both machine learning models 415 and 415′ using a single direction.

FIG. 4C is a block diagram illustrating another machine learning framework 420 that performs two stages of bi-directional passes to train a machine learning model to predict a future appearance of a wound. Machine learning framework 420 includes deep learning architectures 422A, 422B, and 422C (collectively “deep learning architectures 422”). Deep learning architectures 422 each may be a CNN (including U-Net 2D and U-Net 3D), a GAN, a T-Adversarial GAN, a Time Cyclic GAN, or a GAN using privileged information.

In some aspects, deep learning architecture 425A is implemented similarly to deep learning architectures 404A and 404B described above in FIG. 4A and deep learning architecture 411A described above in FIG. 4B. That is, deep learning architecture 422A can be bi-directional and can perform two passes through an image sequence, e.g., a first pass that generates a predicted future image IMGₒᵤₜ from initial images in an image sequence 406, and a second pass that generates a reconstructed first image IMG₁ based on the predicted future image IMGₒᵤₜ and images subsequent to the first image in the image sequence. Deep learning architecture 422A, like deep learning architecture 411A, includes more training data than the example shown in FIG. 4A. However, in the example of FIG. 4C, the additional training data obtained may include a greater number of images from images captured prior to treatment of the wound. In the example illustrated in FIG. 4C, deep learning architecture 422A initially trains machine learning model 425 using images IMG₁-IMG₆. Thus, in the first stage of training, deep learning architecture 422A takes advantage of more image samples to improve the accuracy of machine learning model 425.

During the second stage, deep learning architecture 422B trains machine learning model 425′ using fewer images from images 406. In the example illustrated in FIG. 4C, deep learning architectures 422B and 422C use half the number of images (e.g., IMG1, IMG3 and IMG5). As was the case with the first stage, the second stage of training can be bi-directional with deep learning architecture 422B sharing layers of machine learning model 425′ with deep learning architecture 422C. For example, deep learning architecture 422B trains machine learning model 425′ to generate a predicted future image IMGₒᵤₜ using input images 406 (e.g., IMG₁, IMG₃ and IMG₅) and compares IMGₒᵤₜ to IMG_{label} to determine adjustments to the weights of machine learning model 425′. Additionally, deep learning architecture 422C trains machine learning model 425′ to generate a reconstructed image IMG₁′ using IMGₒᵤₜ, IMG₅ and IMG₃ as input and compares IMG₁′ to IMG₁ to determine adjustments to the weights of machine learning model 425′.

Machine learning framework 420 can impose constraints 424 on the training of machine learning model 425′. For example, machine learning framework 420 can enforce a constraint that certain layers of machine learning model 425′ match the weights of corresponding layers of machine learning model 425. In some aspects, the constraint can be that the weights of the final layer of machine learning model 425′ match the weights of the final layer of machine learning model 425. In some aspects, the constraint can be that the weights of a middle layer of machine learning model 425′ match the weights of a corresponding middle layer of machine learning model 425.

In addition to the aspects discussed above, a further aspect of the disclosure illustrated in FIG. 4C is that despite utilizing fewer samples during an initial phase (e.g., a test phase) of training, the machine learning framework 420 can still produce a machine learning model 425′ that may have the same or similar accuracy as a machine learning model that is trained using more samples.

Like the example illustrated in FIG. 4B, the example illustrated in FIG. 4C, machine learning framework 420 implements bi-directional training (i.e., cyclic training) to train both machine learning model 425 and machine learning model 425′. However, bi-directional training is not a requirement, and in some aspects, machine learning framework 410 can train either or both machine learning models 415 and 415′ using a single direction.

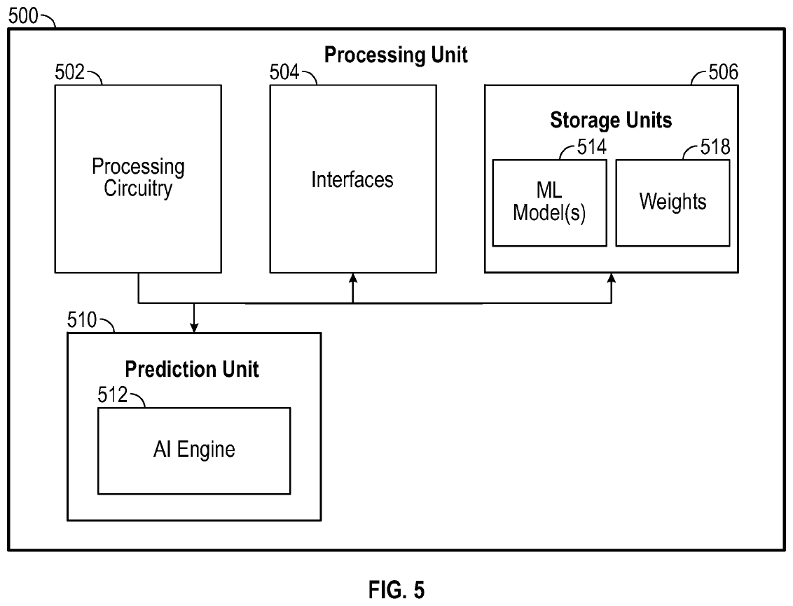

FIG. 5 is a block diagram of an example processing unit of a system for generating a predicted future image of the appearance of a wound, in accordance with at least one example technique described in this disclosure. FIG. 5 is a block diagram illustrating an example processing unit 500, in accordance with at least one example technique described in this disclosure. Processing unit 500 may be an example or alternative implementation of processing unit 104 of FIGS. 1A and 1B. The architecture of processing unit 500 illustrated in FIG. 5 is shown for example purposes only. Processing unit 500 should not be limited to the illustrated example architecture. In other examples, processing unit 500 may be configured in a variety of ways. In the example illustrated in FIG. 5, processing unit 500 includes a prediction unit 510 configured to generate a predicted wound image based on a sequence of input wound images. Prediction unit 510 can include AI engine 512 configured to process the wound image sequences using machine learning model 514 to generate a predicted wound image as output.

In some aspects, machine learning model 514 can include data defining a CNN. In some aspects, machine learning model 514 can include data defining a generative adversarial network (GAN), a T-Adversarial GAN, a U-Net, including U-Net 2D and U-Net 3D.

Processing unit 500 may be implemented as any suitable computing system, (e.g., at least one server computer, workstation, mainframe, appliance, cloud computing system, and/or other computing system) that may be capable of performing operations and/or functions described in accordance with at least one aspect of the present disclosure. In some examples, processing unit 500 represents a cloud computing system, server farm, and/or server cluster (or portion thereof) configured to connect with system 100 via a wired or wireless connection. In other examples, processing unit 500 may represent or be implemented through at least one virtualized compute instance (e.g., virtual machines or containers) of a data center, cloud computing system, server farm, and/or server cluster. In some examples, processing unit 500 includes at least one computing device, each computing device having a memory and at least one processor.

As shown in the example of FIG. 5, processing unit 500 includes processing circuitry 502, at least one interface 504, and at least one storage unit 506. Prediction unit 510, including AI engine 512, may be implemented as program instructions and/or data stored in storage units 506 and executable by processing circuitry 502. Storage unit 506 may store machine learning models 514. Storage unit 506 of processing unit 500 may also store an operating system (not shown) executable by processing circuitry 502 to control the operation of components of processing unit 500. The components, units, or modules of processing unit 500 can be coupled (physically, communicatively, and/or operatively) using communication channels for inter-component communications. In some examples, the communication channels include a system bus, a network connection, an inter-process communication data structure, or any other method for communicating data.

Processing circuitry 502, in one example, may include at least one processor that is configured to implement functionality and/or process instructions for execution within processing unit 500. For example, processing circuitry 502 may be capable of processing instructions stored by storage units 506. Processing circuitry 502, may include, for example, microprocessors, digital signal processors (DSPs), application specific integrated circuits (ASICs), field-programmable gate array (FPGAs), or equivalent discrete or integrated logic circuitry, or a combination of any of the foregoing devices or circuitry.

There may be multiple instances of processing circuitry 502 within processing unit 500 to facilitate processing inspection operations in parallel. The multiple instances may be of the same type, e.g., a multiprocessor system or a multicore processor. The multiple instances may be of different types, e.g., a multicore processor with associated multiple graphics processor units (GPUs).

Processing unit 500 may utilize interfaces 504 to communicate with external systems via at least one network. In some examples, interfaces 504 include an electrical interface configured to electrically couple processing unit 500 to prediction system 102. In other examples, interfaces 504 may be network interfaces (e.g., Ethernet interfaces, optical transceivers, radio frequency (RF) transceivers, Wi-Fi, or via use of wireless technology under the trade “BLUETOOTH”, telephony interfaces, or any other type of devices that can send and receive information. In some examples, processing unit 500 utilizes interfaces 504 to wirelessly communicate with external systems.

Storage units 506 may be configured to store information within processing unit 500 during operation. Storage units 506 may include a computer-readable storage medium or computer-readable storage device. In some examples, storage units 506 include at least a short-term memory or a long-term memory. Storage units 506 may include, for example, random access memories (RAM), dynamic random access memories (DRAM), static random access memories (SRAM), magnetic discs, optical discs, flash memories, magnetic discs, optical discs, flash memories, or forms of electrically programmable memories (EPROM) or electrically erasable and programmable memories (EEPROM). In some examples, storage units 506 are used to store program instructions for execution by processing circuitry 502. Storage units 506 may be used by software or applications running on processing unit 500 to temporarily store information during program execution.

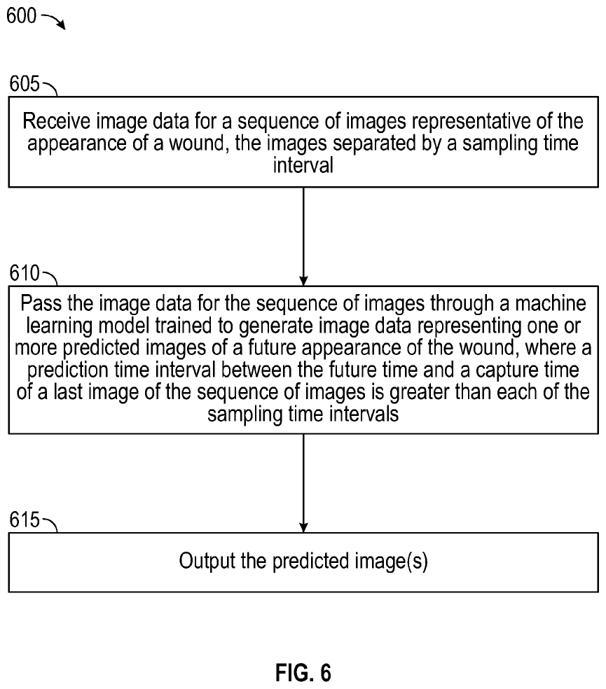

FIG. 6 is a flow diagram illustrating an example operation of a prediction system, in accordance with one or more techniques of this disclosure. The prediction system may receive, by a processing unit comprising one or more processors, image data for a sequence of images representative of an appearance of a wound, the images captured at a plurality of times, each image of the images prior to a final image of the sequence of images separated by a sampling time interval between the image and a next image (605). Next, the prediction system may pass the image data for the sequence of images through a machine learning model trained to generate image data representing a predicted image of a future appearance of the wound at a future time, the machine learning model trained using historical image data, the historical image data comprising a plurality of historical image data sets, each historical image data set of the historical image data sets comprising image data for a historical sequence of images of a corresponding historical wound, wherein a prediction time interval between the future time and a capture time of a last image of the sequence of images is greater than each of the sampling time intervals (610). Next, the prediction system may output the image data representing the predicted image of the future appearance of the wound (615).

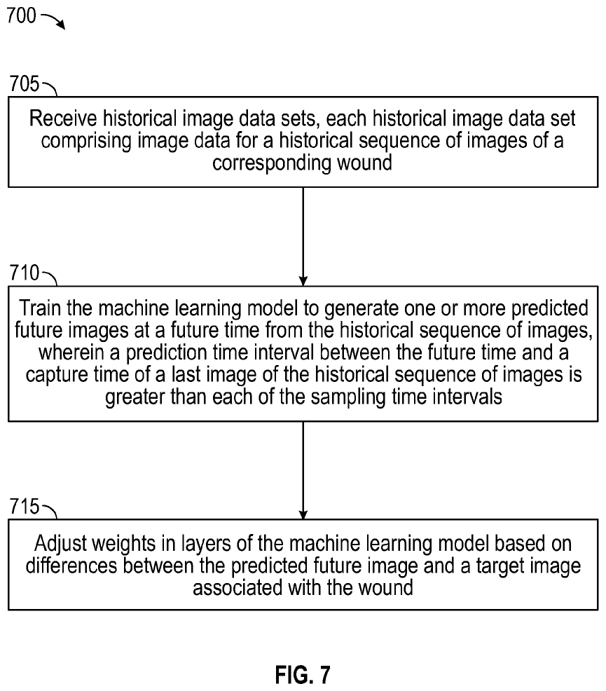

FIG. 7 is a flow diagram illustrating an example operation of a training system, in accordance with one or more techniques of this disclosure. The training system may receive historical image data, the historical image data comprising a plurality of historical image data sets, each historical image data set of the historical image data sets comprising image data for a historical sequence of images of a corresponding wound, each image of the historical sequence of images prior to a final image of the historical sequence of images separated by a sampling time interval between the image and a next image (705). In some aspects, the sampling time interval may be variable. That is, the sampling time interval between the images in the historical sequence need not be uniform across all of the images, and for some images, the sampling interval may be different from the sampling interval for other images. Next, the training system may train the machine learning model to generate a predicted image of a future appearance of the wound at a future time from the historical sequence of images, wherein a prediction time interval between the future time and a capture time of a last image of the historical sequence of images is greater than each of the sampling time intervals (710). Next, the training system may adjust weights in layers of the machine learning model based on differences between the predicted image and a target image associated with the wound (715).

The discussion above has been presented in the context of predicting future images of a wound based on images taken prior to treatment and/or during early stages of treatment of the wound. However, the techniques discussed herein may be applied to the prediction of other properties of a wound and wound treatment. For example, the machine learning model may be trained to predict wound area, wound depth, and/or healing stage based on the input image data, input metadata, or a combination of the two.

Additionally, the techniques discussed herein can be readily applied to other areas as well. For example, the techniques may be applied to images of microbial growth to generate, based on a sequence of images of a microbial colony, a predicted future image of the microbial colony.

The techniques of the disclosure may also be applied to agriculture. Plant growth behavior, like bacterial colony growth and wound healing, can have slow and long progressions. Using the techniques described herein, new cultivars and field regions that would be most resistant to diseases can be predicted using image sequences of fields.

The techniques described in this disclosure may be implemented, at least in part, in hardware, software, firmware or any combination thereof. For example, various aspects of the described techniques may be implemented within at least one processor, including at least one microprocessor, DSP, ASIC, FPGA, and/or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform at least one of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with at least one module and/or unit may be performed by separate hardware or software components or integrated within common or separate hardware or software components.

The techniques described in this disclosure may also be embodied or encoded in a computer-readable medium, such as a non-transitory computer-readable medium or computer-readable storage medium, containing instructions. Instructions embedded or encoded in a computer-readable medium may cause a programmable processor, or other processor, to perform the method (e.g., when the instructions are executed). Computer readable storage media may include RAM, read only memory (ROM), programmable read only memory (PROM), EPROM, EEPROM, flash memory, a hard disk, a CD-ROM, a floppy disk, a cassette, magnetic media, optical media, or other computer-readable storage media. The term “computer-readable storage media” refers to physical storage media, and not signals or carrier waves, although the term “computer-readable media” may include transient media such as signals, in addition to physical storage media.

1. A system comprising:
a memory; and
a processing unit having one or more processors coupled to the memory, the one or more processors configured to execute instructions that cause the processing unit to:
obtain image capture data for a sequence of one or more images representative of an appearance of a wound at a corresponding image capture time, each of the images prior to a final image of the sequence of images separated by a sampling time interval between the image and a next image,
pass the image capture data for the sequence of images through a machine learning model trained to generate image data representing one or more predicted images of the future appearance of the wound, each of the one or more predicted images representative of a future appearance of the wound at a corresponding future time, the machine learning model trained using historical image data, the historical image data comprising one or more historical image data sets, each historical image data set of the one or more historical image data sets comprising image data for a historical sequence of images of an appearance of a corresponding historical wound, wherein a prediction time interval between the future time and a capture time of a last image of the sequence of images is greater than each of the sampling time intervals, and
output the image data representing the one or more predicted images of the future appearance of the wound.

2. The system of claim 1, wherein the image capture data includes metadata identifying a treatment method or one or more treatment method parameters.

3. The system of claim 2, wherein the treatment method parameters include negative-pressure wound therapy (NPWT) parameters.

4. The system of claim 1, wherein the machine learning model is trained bi-directionally, wherein a first direction of training trains the machine learning model to generate the one or more predicted future images from the historical sequence of images and wherein a second direction of training trains the machine learning model to generate a reconstructed first image from the one or more predicted images and images in the historical sequence of images subsequent to the first image.

5. The system of claim 4, wherein layers in the machine learning model are shared by the first direction of training and the second direction of training.

6. The system of claim 4, wherein:
the machine learning model comprises a second machine learning model;
a first machine learning model is trained prior to the second machine learning model using a first training image data set that includes a first subset of images of the historical sequence of images captured during a sampling period associated with the historical wound images and a second subset of images captured after the sampling period; and
the second machine learning model is constrained to include one or more layers of the first machine learning model.

7. The system of claim 6, wherein the first machine learning model is trained bi-directionally.

8. The system of claim 6, wherein the one or more layers comprise a final layer, penultimate layer, or one or more mid-level layers.

9. The system of claim 1, wherein:
the machine learning model comprises a second machine learning model;
a first machine learning model is trained prior to the second machine learning model using a first training image data set that includes a first subset of images of the historical sequence of images captured during a sampling period associated with the historical wound and a second subset of images captured during the sampling period, wherein a number of images in the first subset of images is greater than the number of images in the second subset of images; and
the second machine learning model is constrained to use one or more layers of the first machine learning model.

10. The system of claim 9, wherein the first machine learning model is trained bi-directionally.

11. The system of claim 1, wherein the prediction time interval is greater than an input time interval associated with the sequence of images.

12. The system of claim 1, wherein the machine learning model is trained using historical metadata corresponding to the historical image sequence, and wherein the processing unit is further configured to:
obtain metadata comprising wound properties of the wound corresponding to the sequence of images, the wound properties comprising one or more of wound area, wound depth, or wound healing stage;
pass the metadata through the machine learning model to generate predicted metadata for the wound at the corresponding future time; and
output the predicted metadata.

13. A method comprising:
obtaining, by a processing unit comprising one or more processors, image capture data for a sequence of one or more images representative of an appearance of a wound at a corresponding image capture time, each of the images prior to a final image of the sequence of images separated by a sampling time interval between the image and a next image;
passing the image capture data for the sequence of images through a machine learning model trained to generate image data representing one or more predicted images of the future appearance of the wound, each of the one or more predicted images representative of a future appearance of the wound at a corresponding future time, the machine learning model trained using historical image data, the historical image data comprising one or more historical image data sets, each historical image data set of the one or more historical image data sets comprising image data for a historical sequence of images of an appearance of a corresponding historical wound, wherein a prediction time interval between the future time and a capture time of a last image of the sequence of images is greater than each of the sampling time intervals; and
outputting the image data representing the one or more predicted images of the future appearance of the wound.

14. The method of claim 13, wherein the machine learning model is trained using a weighted loss that assigns a first weight to a first image that is less than a second weight assigned to a second image having a corresponding predicted future time that is later than the predicted future time corresponding to the first image.

15. The method of claim 13, wherein the machine learning model is trained bi-directionally, wherein a first direction of training trains the machine learning model to generate the one or more predicted images from the historical sequence of images and wherein a second direction of training trains the machine learning model to generate a reconstructed first image from the one or more predicted images and images in the historical sequence of images subsequent to the first image.

16. The method of claim 15, wherein layers in the machine learning model are shared by the first direction of training and the second direction of training.

17. The method of claim 15, wherein:
the machine learning model comprises a second machine learning model;
a first machine learning model is trained prior to the second machine learning model using a first training image data set that includes a first subset of images of the historical sequence of images selected from a sampling period associated with the wound and a second subset of images selected from images of the wound captured after the sampling period; and
the second machine learning model is constrained to include a layer of the first machine learning model during a training phase of the second machine learning model.

18. The method of claim 17, wherein the first machine learning model is trained bi-directionally.

19. The method of claim 17, wherein the layer comprises a final layer.

20. The method of claim 15, wherein:
the machine learning model comprises a second machine learning model;
a first machine learning model is trained prior to the second machine learning model using a first training image data set that includes a first subset of images of the historical sequence of images captured during a sampling period associated with the historical wound and a second subset of images captured during the treatment period of the wound, wherein a number of images in the first subset of images is greater than the number of images in the second subset of images; and
the second machine learning model is constrained to use a layer of the first machine learning model.

21-32. (canceled)
